(19) United States
(12) Patent Application Publication
(10) Pub. No.: US 2018/0279887 A1
(43) Pub. Date: Oct. 4, 2018

(54) DEVICE AND METHOD FOR NON-INVASIVE MEASUREMENT OF SUBDIAPHRAGMATIC AORTIC FLOW IN A SMALL LABORATORY MAMMAL

(71) Applicant: UNIVERSITE GRENOBLE ALPES, St. Martin d'Hères (FR)

(72) Inventors: Pierre BACONNIER, Grenoble (FR); François BOUCHER, Grenoble (FR); Pierre-Yves GUMERY, Grenoble (FR)

(73) Assignee: UNIVERSITE GRENOBLE ALPES, St. Martin d'Hères (FR)

(21) Appl. No.: 15/754,802

(22) PCT Filed: Aug. 26, 2016
(86) PCT No.: PCT/FR2016/052130

§ 371 Date: Feb. 23, 2018

(30) Foreign Application Priority Data
Aug. 28, 2015 (FR) 1557992

(51) Int. Cl. A61B 5/0295; A61B 5/00; A61B 5/0402; A61B 7/04; A61B 5/0265; A61B 5/0205

(52) U.S. Cl. CPC A61B 5/0295; A61B 5/6804; A61B 5/0402; A61B 7/04; A61B 5/7278; A61B 5/0806; A61B 5/0205; A61B 5/7225; A61B 2503/40; A61B 2503/42; A61B 2562/0219; A61B 5/0265

(57) ABSTRACT

A device non-invasively measures aortic flow in the subdiaphragmatic region in a small laboratory mammal. The device includes a device for plethysmography of the thorax by inductance measurement, having two electrically conducting, extendible coils rigidly attached to an elastic garment that is adjustable to the torso of the mammal, a device for acquiring the signal from variation in the cross-section of each coil, and a processor configured to calculate the instantaneous subdiaphragmatic aortic flow of the mammal from the signals from the variation in cross-section of each coil and from a functional model of the cardiorespiratory system. The exchanges of blood between the thorax and the rest of the body of the mammal include an output of blood via the abdominal aorta in the subdiaphragmatic region and an input of blood via the inferior vena cava.

### CROSS-REFERENCE TO RELATED APPLICATIONS

This application is a National Phase Entry of International Patent Application No. PCT/FR2016/052130, filed on Aug. 26, 2016, which claims priority to French Patent Application Serial No. 1557992, filed on Aug. 28, 2015, both of which are incorporated by reference herein.

### TECHNICAL FIELD

The present invention relates to a device for non-invasive measurement of the sub-diaphragmatic aortic flow in a small laboratory mammal.

### BACKGROUND

Cardio-respiratory exploration in humans or animals has been implemented for many years now. In small laboratory mammals, even if they have evolved over time, cardio-respiratory exploration techniques are generally invasive. In 1860 Chauveau and Lortet were the first to describe apparatus (haemodromograph) for invasive measuring of variations of flow speed of blood in a live mammal.

In 1947, Timm proposed a cinematoradiography technique at low speed for evaluating the travel speed of a bubble of a hydrophobic contrast product injected into the aorta artery of dogs and cats. This technique has since been improved with use of cinematography at high speed for measuring the travel speed of fat globules in sufficiently transparent vessels. This technique proved applicable to the aorta of rabbits [1].

Initial use of an electromagnetic flow meter for measuring aortic blood flow in vivo was made in 1956 [2], while the first methods for measuring flow by dilution of dye or by thermodilution started to appear in studies on large mammals or in humans. The latter provide an average value of the cardiac output only over a long time relative to the duration of the cardiac cycle.

Several techniques currently enable measurement of aortic blood flow in laboratory glires (placental mammals) by direct positioning of a sensor around the aorta artery, also called an aortic ring. This sensor is connected either to a device for acquisition by a wired system, in which case physiological measurements of aortic flow are taken under general anaesthesia of the animal, or by a telemetric device which enables acquisition of the aortic flow in the conscious animal, after a post-operative recovery period. The sensors most used on placental mammal models up to the 2000s were electromagnetic sensors [3-6] which have been progressively replaced by sensors whereof the operation is based on measuring ultrasound transit time (“transit time ultrasound technology”) [7-10].

Non-invasive exploration of the cardiac function in small animals is performed today only by methods (PET, MRI, CT scan, Echography, pulsed Doppler) [11-14] extremely difficult to execute and restricting for animals. Also, some of these methods (PET, CT scan and some MRI protocols) need catheterization. An inductance measurement solution based on sub-cutaneous electrodes (which is therefore invasive) [15] has not resulted in commercial development. There are telemetry solutions based on implantable (and therefore invasive) systems such as the product marketed by the company DSI™ [16].

### SUMMARY

An aim of the invention is continuous measuring of the flow of the aorta in the sub-diaphragmatic region in the small laboratory mammal, such as small rodent and lagomorph, non-invasively, non-restraining and in accordance with regulatory restrictions. In keeping with the invention, a device for non-invasive measurement of the aortic flow in the sub-diaphragmatic region in a small laboratory mammal is proposed, characterized in that it comprises:
a device for thorax plethysmography by inductance measurement comprising two electrically conductive extensible coils integral with an elastic garment adjustable to the trunk of said mammal,
a device for acquiring the signal of variation in cross-section of each coil,
a processor configured to calculate the instantaneous sub-diaphragmatic aortic flow of said mammal, from said signals of variation in cross-section of each coil and from a functional model of the cardio-respiratory system according to which the exchanges of blood between the thorax and the rest of the body of said mammal consist of output of blood via the abdominal aorta in the sub-diaphragmatic region and input of blood via the inferior vena cava.

The metrological performances of this measuring by thoracic inductive plethysmography (TIP) are dictated by the levels of volumes to be measured, from a few tens of microliters for blood volumes pushed in the aorta with each heartbeat in the small animal to a few milliliters in lagomorphs, and by the dynamic characteristics of the physiological function of interest (cardiac frequency of 2 to 10 Hz, according to species). These metrological restrictions are more drastic than those dictated by respiratory inductive plethysmography (RIP), for which volumes are a few tens of milliliters and lowest physiological frequencies (<1 Hz).

According to other advantageous features, considered singly or in combination:
the diameter of each coil in the free state is between 2 and 15 cm;
the space between said coils is between 0.5 and 5 cm;
the coils are arranged in zigzag and, in the free state, the spatial period of the zigzags is between 0.5 and 1.5 cm;
the range of said zigzags is between 0.5 and 3 cm.
   “Free state” means the state of the plethysmography device when the animal does not wear the garment.

Also, the device can further comprise at least one of the following elements:
a coil integral with said garment intended to surround the abdomen of the mammal so as to perform respiratory plethysmography measurement by inductance measurement;
an electrocardiographic sensor integral with said garment;
an accelerometer integral with said garment;
a microphone integral with said garment, said microphone being intended to record the cardiac sounds of the mammal.

Another aim of the invention relates to a method for non-invasive measurement of the aortic flow in the sub-diaphragmatic region in a small laboratory mammal, characterized in that it comprises:
acquisition of a thoracic plethysmography signal by inductance measurement by means of two electrically conductive extensible coils integral with an elastic garment adjustable to the trunk of said mammal,
extraction, by means of a processor, from said plethysmographic signal, of the cardiac component of the variations in volume of the thorax and deduction of the sub-diaphragmatic aortic flow on the basis of a functional model of the cardio-respiratory system according to which the exchanges of blood between the thorax and the rest of the body of said mammal consist of output of blood via the abdominal aorta in the sub-diaphragmatic region and input of blood via the inferior vena cava.

Particularly advantageously, the sub-diaphragmatic aortic flow D_{Ao, sd} can be determined from the formula:

D_{Ao, sd}=−dV_{bt}(t)/dt+Q_{c}

where t is the time, V_{bt}(t) is the cardiac component of the variations in volume of the thorax and Q_{c} is the average cardiac output of said mammal.

Advantageously, the average cardiac output Q_{c} is determined by calculating the average of the signal dV_{bt}(t)/dt on the last third of the cardiac cycle of said mammal and by assigning said average value to the average cardiac output.

According to embodiments implemented separately or in combination:
a respiratory plethysmography measurement is further acquired by inductance measurement of said mammal;
an electrocardiogram of said mammal is further acquired and the electrocardiographic signal is taken into account in the extraction of the cardiac component of the variations in volume of the thorax;
an accelerometric signal representative of an activity of said mammal is further acquired and said accelerometric signal is taken into account in extraction of the cardiac component of the variations in volume of the thorax;
the cardiac sounds of said mammal are further recorded by means of a microphone and said sound signal is taken into account in the extraction of the cardiac component of the variations in volume of the thorax.
   According to an advantageous embodiment, said method is implemented on a non-restrained conscious mammal.

### BRIEF DESCRIPTION OF THE DRAWINGS

Other features and advantages of the invention will emerge from the following detailed description in reference to the appended drawings, in which:

FIG. 1 is a graphic representation of the functional physiological model implemented in the invention;

FIG. 2 is a graphic representation of the signals used for determining the aortic flow in the sub-diaphragmatic region; and

FIG. 3 is a main diagram of a device according to the invention.

### DETAILED DESCRIPTION

The invention is based on carrying out plethysmography of the thorax and extraction, from this measurement, of a cardio-circulatory functional variable: the flow of the sub-diaphragmatic aorta. As for plethysmography, several technologies can be implemented. The technology described here is technology by inductance measurement which is used conventionally in respiratory inductive plethysmography (RIP) and applied here to the variations in volume of the thorax of cardiac and respiratory origin (TIP).

The subjects targeted by such cardio-circulatory functional investigation are small laboratory mammals such as rodents (mice, rats, guinea pigs, hamsters, gerbils, ferrets) and lagomorphs (rabbits). In general, these animals present a mass of under 6000 g. For taking measurements, the animal can be anaesthetised or conscious. In the latter case, extra measurements are necessary to take into account the artefacts linked to the activity of the animal.

In all cases, measurements are made non-invasively, i.e., they need no shaving of the animal or intervention of a surgical nature. The cardiac and respiratory activities modify the volumes of the thorax and abdomen of the subject. Placing two inductance-measuring coils in two regions of the thorax of the animal can estimate variations in volume of the thorax over time by a linear combination of the variations in cross-section estimated in these two regions by each of the two coils [17].

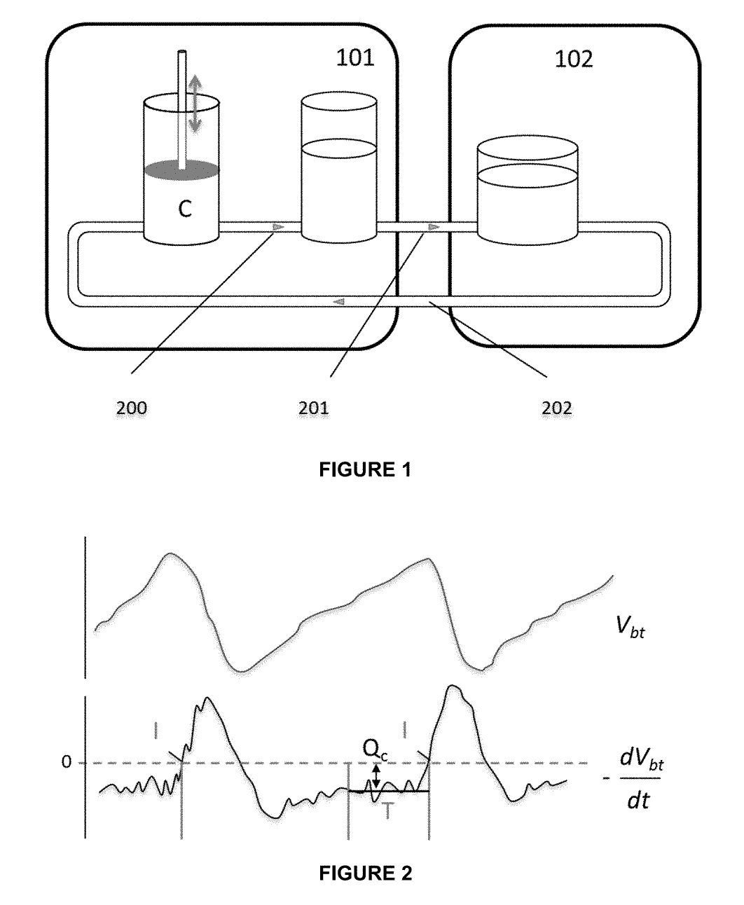

FIG. 1 is a graphic representation of the functional physiological model on which calculation of the sub-diaphragmatic aortic flow is based. The cardiac pump C pushes the systolic ejection volume into a cluster of communicating compartments: the thoracic compartment 101 via the thoracic aorta 200, then the rest of the organism 102, mainly via the abdominal aorta 201. The return of blood to the thorax occurs via the inferior vena cava 202.

According to this model, it is assumed that the variations in volume of the thorax of cardiac origin are linked to the rush of blood out of the thorax via the abdominal aorta during the systole (contraction of the left ventricle) then to the venous return, which is constant. According to the functional physiological model of FIG. 1, the cardiac component of the variation in volume of the thorax, noted V_{bt}, is the result of the dynamic imbalance between the blood flow leaving the thorax and the blood flow entering it. The functional physiological model as proposed reduces exchanges of blood between the thorax and the rest of the body to those passing through the abdominal aorta, in the sub-diaphragmatic region (output) and the inferior vena cava (input).

If the following instantaneous variables are defined:

D_{A0, sd}(t)=aortic flow in the sub-diaphragmatic region,

D_{VCinf}(t)=flow of the inferior vena cava in the same region,

the equation (1) connecting the derivative according to the time of the cardiac component of the volume of the thorax and flows of the two relevant vessels can be written:

d_{Vbt}(t)/dt=D_{VCinf}(t)−D_{Ao,sd}(t) (1)

The flow of the vena cava is constant, equal to the average cardiac output (Q_{c}), therefore:

D_{Ao, sd}(t)=−dV_{bt}(t)/dt+Q_{c} (2)

The estimation of Q_{c} is made possible because the aortic flow in the sub-diaphragmatic region is cancelled out during the latter part of the cardiac cycle.

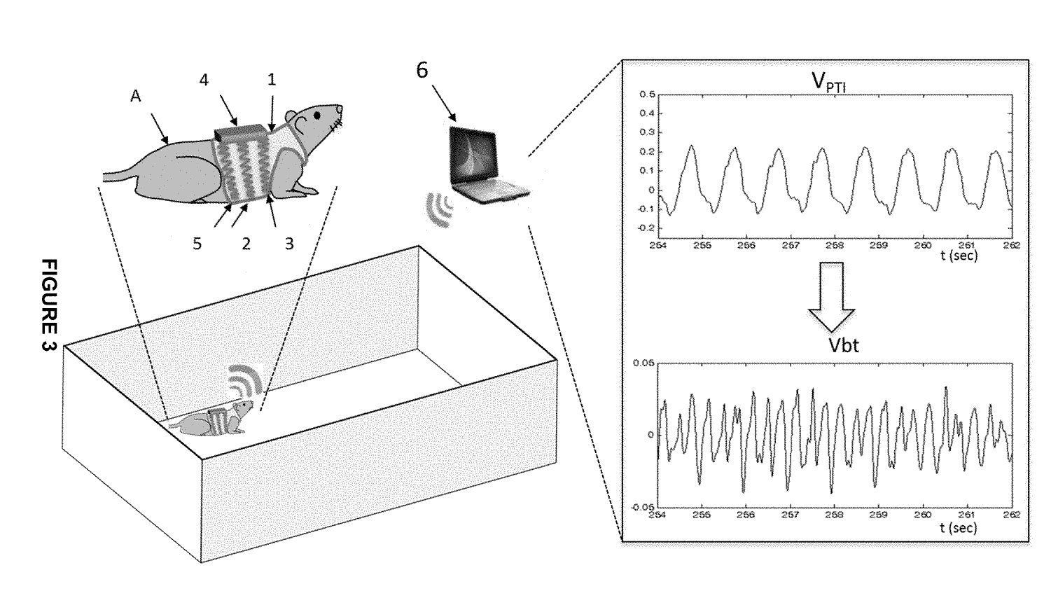

FIG. 2 is a graphic representation of the signals used in calculating the signal V_{bt}(t) and its derivative dV_{bt}(t)/dt. The signal V_{bt}(t) increases periodically in diastole and decreases sharply at each systole. To determine the aortic flow in the sub-diaphragmatic region, by means of a processor, an algorithm is run whereof the different steps are the following:
detection of the start of the ejection phase as being the intersection I of [−dV_{bt}(t)/dt] with the axis of the abscissa before the positive peak of maximal range;
calculation of the average of the signal [dV_{bt}(t)/I] on the last third T of the cycle; and
assignation of this value at Q_{c} which allows calculation of D_{Ao, sd}(t) according to the equation (2).

The measuring device is a garment which can be integrated into a vest adjusted on the body of the animal. It is ensured that the two coils, arranged in zigzag to accompany deformations of the cross-sections which they enclose, are positioned around two regions of the animal's thorax. The garment is advantageously made of an extensible material to fit the body of the animal tightly. The garment typically has tubular form, with optionally holes for passage of the feet of the animal. The coils are fixed to the garment for example by sewing.

For animals of this size, the diameter of the coils in the free state is typically between 2 and 15 cm. Also, the space between the two coils is typically between 0.5 and 5 cm. As for the range and the spatial period of the zigzags of the coils, they are respectively in the order of 0.5 to 5 cm and of 0.5 to 3 cm. Relative to the RIP, used conventionally in humans or large animals, the nominal value of the inductance of the coils of the present device intended for small laboratory mammals is less by a factor of about 10, given its size.

There are different models of garments and configuration of coils depending on the weight range of the animal and/or the species in question. Each coil forms part of an oscillator whereof the frequency (which is linked to the inductance value of the corresponding coil) is measured over time. For this reason, a counting method is implemented.

The spectral content of the cardiac component of interest needs a calibration frequency of the order of 100 Hz. To have such a calibration frequency, a rapid counting tool must be used (in the order of 200 MHz). Also, this tool must be adaptive to deal with the major changes in thoracic cross-section caused by postural modifications of the animal. The measuring device also comprises a processor configured to run the algorithm for extracting from the signal V_{bt}(t) the aortic flow in the sub-diaphragmatic region according to equation 2.

To isolate the cardiac component of the signal for variation in volume of the thorax, the ventilatory component of the signal must be filtered. Conventional filtering techniques with a low cut-off frequency equal to or greater than the average cardiac frequency have been discarded in favour of digital processes: scale approaches (wavelets, empirical mode decomposition) and/or adaptive filterings. Also, the measuring device must be calibrated.

To this end, direct measurement is made of the sub-diaphragmatic aortic flow on an animal of the species for which the device is intended. Such a direct measurement can for example be made by means of an aortic ring which is a device implanted in the sub-diaphragmatic region around the aorta of the animal and which enables measuring of flow by transit time of ultrasound. Reference measurements per range of species and weight are made on anaesthetised animals equipped with the device. The device is calibrated by least-squares fitting of the parameters of the linear combination for estimating the sub-diaphragmatic aortic flow from two cross-sectional measurements.

It should be noted that due to its invasive character such a calibration method is not adapted to humans. However, in small laboratory mammals, direct measurement of the sub-diaphragmatic aortic flow can be implemented on an animal of a given species and used to calibrate the device intended to be used on the animals of the same species.

FIG. 3 illustrates an embodiment for measuring the aortic flow in the sub-diaphragmatic region according to the method described above. The vest 1 is a non-invasive device for preserving the integrity of the animal A by way of plethysmographic measuring by varying the inductance of two coils 2, 3 located in the region of the thorax. The vest also carries an electronic circuit 4 intended to condition signals. An additional coil 5 can optionally be added to the abdominal region to enable plethysmographic measuring of ventilatory order (RIP). The positioning and configuration of coils (range and spatial period of the zigzags) are determined by the abovementioned physiological model and standardized according to morphological criteria (weight ranges, relevant species).

The device further comprises a processor, for example forming part of a computer 6. Said processor is configured to calculate, from signals of variation in cross-section of the coils, the variation in thoracic volume (V_{PTI} signal) and extract the cardiac component (V_{bt} signal) from which the sub-diaphragmatic aortic flow is determined.

By way of particular advantage, the plethysmography data are transmitted continuously to the processor by telemetry. This particularly minimizes stress imposed on the animal, or even implements the invention on a non-restrained conscious animal. But the invention is not limited to this embodiment and if needed can comprise a wire link between the plethysmographic device and the processor.

Optionally, the vest can carry one or more sensors other than the cardio-circulatory plethysmographic device. So, especially when the animal is conscious and non-restrained, the vest can carry an accelerometer for considering artefacts linked to the activity of the animal in the extraction algorithm. The accelerometric data are used in filtering movement artefacts (noise reference).

Also, the vest can carry an electrocardiographic sensor. Finally, the vest can also carry a microphone adapted to record the cardiac sounds of the animal. Electrocardiographic, sonocardiographic and RIP data further integrate cardio-respiratory exploration into the device.

### REFERENCES

[1] McDonald D. A., “The velocity of blood flow in the rabbit aorta studied with high-speed cinematography”. J. Physiol. 1952, 118:328-339.
[2] Spencer M. P. and Denison A. B., “The Aortic Flow Pulse as Related to Differential Pressure”. Circ. Res. 1956, 4:476-484.
[3] U.S. Pat. No. 2,808,723
[4] U.S. Pat. No. 3,592,187
[5] U.S. Pat. No. 4,346,604
[6] Berthonneche C., Sulpice T., Boucher F., Gouraud L., de Leiris J., O'Connor S. E.,
Herbert J. M. and Janiak P., “New insights into the pathological role of TNF—. in early cardiac dysfunction and subsequent heart failure following myocardial infarction in rats” Am. J. Physiol. 2004, 287:H340-H350.
[7] U.S. Pat. No. 4,337,667
[8] U.S. Pat. No. 6,098,466
[9] U.S. Pat. No. 7,469,598
[10] Hoffman A., Grossman E., Ohman K. P., Marks E. and Harry R. Keiser H. R., “Endothelin Induces An Initial Increase in Cardiac Output Associated with Selective Vasodilation in Rats” Life Sci. 1989, 45(3):249-255.
[11] Xiong G., Paul C., Todica A., Hacker M., Bartenstein P. and Boning G., “Noninvasive image derived heart input function for CMRgIc measurements in small animal slow infusion FDG PET studies”. Phys. Med. Biol. 2012, 7; 57(23):8041-59. doi: 10.1088/0031-9155/57/23/8041. Epub 2012 Nov. 16. PubMed PMID: 23160517.
[12] Bible E., Dell'Acqua F., Solanky B., Balducci A., Crapo P. M., Badylak S. F., Ahrens E. T. and Modo M., “Non-invasive imaging of transplanted human neural stem cells and ECM scaffold remodeling in the stroke-damaged rat brain by (19)F—and diffusion—MRI”. Biomaterials. 2012, 33(10):2858-71.
[13] Dinkel J., Bartling S. H., Kuntz J., Grasruck M., Kopp-Schneider A., Iwasaki M., Dimmeler S., Gupta R., Semmler W., Kauczor H. U. and Kiessling F., “Intrinsic gating for small-animal computed tomography: a robust ECG-less paradigm for deriving cardiac phase information and functional imaging”. Circ. Cardiovasc. Imaging. 2008, 1(3):235-43.
[14] Nakamura T, Matsumuro A, Kuribayashi T, Matsubara K, Shima M, Shimoo K, Katsume H, Nakagawa M., “Echocardiographic determination of stroke volume during rapid atrial pacing and volume loading in normal rats”. Cardiovasc. Res. 1992, 26(8):765-9.
[15] Gotshall R. W., Breay-pilcher J. C. and Boelcskevy B. D., “Cardiac output in adult and neonatal rats utilizing impedance cardiography”. Am. J. Physiol. 1987, 253(22): H1298-H1304.
[16] http://www.datasci.com/products/implantable-telemetry/[17]
[17] Konno K. and Mead J. “Measurement of separate volume changes of rib cage and abdomen during breathing”. J. Applied Physiol. 1967, 22(3):407-22.

1. A device for non-invasive measurement of an aortic flow in a sub-diaphragmatic region in a small laboratory mammal, comprising:
a thorax plethysmography by inductance measurement device comprising two electrically conductive extensible coils integral with an elastic garment adjustable to a trunk of said mammal;
a device configured to acquire a signal of variation in cross-section of each coil; and
a processor configured to calculate instantaneous sub-diaphragmatic aortic flow of said mammal, from said signals of variation in cross-section of each coil and from a functional model of a cardio-respiratory system according to which exchanges of blood between a thorax and a remainder of a body of said mammal include output of said blood by an abdominal aorta in a sub-diaphragmatic region and input of said blood via an inferior vena cava.

2. The device according to claim 1, wherein a diameter of each of said coils in a free state is at least one of: between 2 and 15 cm or a space between said coils is between 0.5 and 5 cm.

3. The device according to claim 1, wherein said coils are arranged in zigzag and, in a free state, at least one of: a spatial period of said zigzags is between 0.5 and 1.5 cm or a range of said zigzags is between 0.5 and 3 cm.

4. The device according to claim 1, further comprising a coil integral with said garment configured to surround an abdomen of said mammal so as to perform a respiratory plethysmography measurement by an inductance measurement.

5. The device according to claim 1, further comprising an electrocardiographic sensor integral with said garment.

6. The device according to claim 1, further comprising an accelerometer integral with said garment.

7. The device according to claim 1, further comprising a microphone integral with said garment, said microphone being configured to record cardiac sounds of the mammal.

8. A method for non-invasive measurement of an aortic flow in a sub-diaphragmatic region in a small laboratory mammal, comprising:
acquiring a thoracic plethysmography signal by inductance measurement by two electrically conductive extensible coils integral with an elastic garment adjustable to a trunk of said mammal; and
extracting, by a processor, from said plethysmographic signal, a cardiac component of variations in volume of a thorax and deducing sub-diaphragmatic aortic flow on a basis of a functional model of a cardio-respiratory system according to which exchanges of blood between said thorax and a remainder of a body of said mammal include output of blood via an abdominal aorta in a sub-diaphragmatic region and input of blood via an inferior vena cava.

9. The method according to claim 8, wherein the sub-diaphragmatic aortic flow is determined from a formula:
D_{Ao, sd}(t)=−dV_{bt}(t)/dt+Q_{c}
where D_{Ao, sd} is said sub-diaphragmatic aortic flow, t is time, V_{bt}(t) is a cardiac component of variations in volume of said thorax and Q_{c} is an average cardiac output of said mammal.

10. The method according to claim 9, wherein an average cardiac output is determined by calculating an average of a signal dV_{bt}(t)/dt on a last third of a cardiac cycle of said mammal and by assigning said average value to said average cardiac output.

11. The method according to claim 8, wherein a respiratory plethysmography measurement is further acquired by inductance measurement of said mammal.

12. The method according to claim 8, wherein an electrocardiogram of said mammal is further acquired and an electrocardiographic signal is taken into account in an extraction of said cardiac component of variations in volume of said thorax.

13. The method according to claim 8, wherein an accelerometric signal representative of an activity of said mammal is further acquired and said accelerometric signal is taken into account in extraction of said cardiac component of variations in volume of said thorax.

14. The method according to claim 8, wherein cardiac sounds of said mammal are further recorded by a microphone and a sound signal is taken into account in extraction of said cardiac component of variations in volume of said thorax.

15. The method according to claim 8, implemented on said mammal which is non-restrained and conscious.
